(19) United States
(12) Patent Application Publication
(10) Pub. No.: US 2021/0244390 A1
(43) Pub. Date: Aug. 12, 2021

(54) SAMPLE REMOVAL ASSEMBLY FOR GYNECOLOGICAL EXAMINATIONS

(71) Applicant: Mediscene AG, Basel (CH)

(72) Inventor: Bernhard Rinderknecht, Basel (CH)

(21) Appl. No.: 16/972,137

(22) PCT Filed: Jun. 4, 2019
(86) PCT No.: PCT/EP2019/064534

§ 371 Date: Dec. 4, 2020

(30) Foreign Application Priority Data
Jun. 4, 2018 (DE) 10 2018 208 769.5

(51) Int. Cl. A61B 10/02; A46B 3/18; A46D 1/00

(52) U.S. Cl. CPC A61B 10/0291; A46B 3/18; A46B 2200/40; A61B 2010/0216; A46D 1/0207

(57) ABSTRACT

A gynecological cervical or vaginal sample removal instrument has, at the cranial end, a plurality of brushes that are fixed to a wire assembly extending in the axial direction. The brushes are made of at least 600 plastic filaments with a diameter between 350 μm and 900 μm and an axial line density of at least 600 filaments over a maximum axial extension of 3 cm.

### CROSS-REFERENCE TO RELATED APPLICATIONS

The present application is the national stage of International Pat. App. No. PCT/EP2019/064534 filed Jun. 4, 2019, and claims priority under 35 U.S.C. § 119 to DE 10 2018 208 769.5, filed in Germany on Jun. 4, 2019, the content of each of which are incorporated herein by reference in their entireties.

### FIELD OF THE INVENTION

The present invention relates to the field of gynecological examinations.

### BACKGROUND

In many gynecological examinations and screening procedures, it is necessary to collect cervical or vaginal smears and to examine the collected material in a laboratory.

A key role in preventive medicine is the prevention of cervical cancer, which is to date the most effective primary preventive screening measure of all, in which cells are removed and are used to identify the microscopically easily detectable cancer precursors (dysplasias) of the cervix. Since the 1950s, it has been customary for this purpose to carry out tests called PAP tests after the first observations described by Professor George Papanicolaou in 1928. In this cell smear test, cells are taken from the woman's cervix, stained and examined under a microscope. The person carrying out the examination smears the cells onto a glass slide and fixes them with an alcohol solution. The material (cells and simultaneously collected mucopolysaccharide-containing mucosal substance) can also be transferred into a small container that is filled for example with ethanol, isopropanol or methanol and that fixes the cell material, prevents cell degeneration and preserves the cell material for further processing. Such samples can be examined, not only cytologically by microscopy, but also at a molecular biological level, e.g., for HPV human papillomavirus identifiers, biomarkers, e.g., p16 and Ki-67. The various solvents have to be chosen such that the collected material does not form lumps and such that the subsequent analysis can be performed as intended.

The examinations in question are nowadays carried out routinely and in large numbers.

In order to obtain the correct material in the greatest possible quantity for the subsequent tests, the associated pre-analytical error of the method has to be minimized, which corresponds to an increased method sensitivity. The two determining factors here are, firstly, the material being collected from the correct site and, secondly, the quantity of material.

The anatomy of the removal site, i.e., the cervix, is subject to macroscopic and microscopic, life-long transformations that are virtually unique to the human body. Through the biological force of reproduction, the cervix uteri experiences very considerable changes during a woman's lifetime, especially influenced by the two main female hormone groups, i.e., estrogens and gestagens. Microscopically, the cervix is covered by two layers of epithelial cells, namely the thicker, multi-layered squamous epithelium and the single-layer cylindrical epithelium. The zone where the two epithelia meet and are partially converted into each other by a process called metaplasia is called the transformation zone, conversion zone, transition zone or junction zone. This zone is the region that is relevant in the prevention of cervical cancer. It is generally there that the cancerous process takes place, i.e., the appearance of cancer precursors (dysplasias). Moreover, on account of its “thin-skinned” vulnerability, the single-layer cylindrical epithelium cranially adjoining the transformation zone is especially the preferred point of entry and residence of certain infectious microorganisms. Under the effect of the estrogens in puberty and adolescence, the transformation zone in girls “migrates” from the interior of the cervix (endocervix) to the ectocervix (ectopia, ectropion), and in pregnancy it often migrates still further (pregnancy ectopia) in order then to return to the endocervix after menopause. In addition, the macroscopic structure and form of the cervix are subject to some considerable changes of shape as a result of labor, surgery, e.g., cervical incisions, and the atrophy (tissue ageing) accelerated by estrogen deficiency.

This can lead to problems in subsequent examinations of the collected samples. If the sample is collected from an unsuitable site, in particular from too far inside, there is the danger of obtaining incorrect laboratory results. It is desirable here to have an instrument that can be used for all anatomical circumstances, i.e., independently of the patient's age for example.

Irrespective of this, the swab instrument should permit correct removal of material as far as possible in all situations and should collect as much material as possible. By virtue of its design, the instrument should at the same time permit standardized “fool-proofed” removal of material, which in principle will allow paramedical personnel without basic knowledge of anatomy to perform safe and valid removal of sample material.

In the vast majority of cases, a smear test is found to be an unpleasant and sometimes even painful experience. It is therefore advantageous to collect the material in such a way that the actual intervention on the patient is brief and does not have to be repeated. Moreover, as has been mentioned, it should also be possible for less experienced physicians and paramedical personnel to collect the required sample material safely and without any problems.

Sample removal for mass testing also has to be carried out using cost-effective instruments, which are nowadays exclusively, and in the present case too, disposable instruments.

The PAP test after Papanicolaou was originally carried out with vaginal cells that were collected using a pipet. The Ayre spatula and then the cytobrush developed by Nils Stormby subsequently led to a significant increase in sensitivity. This brush is typically used with a speculum and under visual monitoring. However, it can happen that the brush is not moved to the correct location, and in particular it can happen that it is pushed in too far by inexperienced physicians. Moreover, for the collection of samples, the use of a spatula is additionally recommended in order to collect material from all anatomically relevant regions.

U.S. Pat. No. 3,881,464 discloses a sample removal device that is used to collect a sample from the cervix and that is said to be suitable for both cytological and histological tests, for which purpose a cervical sample with tissue and cellular material is obtained by bringing the cervix into contact with a generally conical brush. The bristles of this brush are intended to be soluble in the later used solvent, but insoluble in conventional fixing agents for cells. The generally conical shape is intended to be achieved by the bristles generally extending radially away from a shaft. At the caudal end, i.e., the end toward the outside of the patient, this diameter measures approximately 2.5 cm. The diameter decreases over a distance of ca. 2 cm to 0.5 cm with an approximately linear reduction, and a section measuring ca. 1 cm in length and having an approximately constant diameter of 0.5 cm adjoins at the cranial end (the end toward the inside of the patient).

U.S. Pat. App. Pub. No. 2009/0240164 A1 discloses a brush for gynecological examinations, said brush being intended for use in the uterus. It mentions in the introduction that different brushes have different limitations, for example in the sense that they do not collect enough cells, that their use causes discomfort, that the depth of insertion can be uncertain, etc.

DE 699 28 281 T2 discloses a smear brush. The introduction mentions a smear sample being taken from the cervix, and reference is made to the Medscand cytobrush. It argues that a disadvantage of the latter is that nylon hairs are cut to the correct length using a blunt tool, which would cause pointed hair ends, and that the wire is stiff. DE 699 28 128 T2 proposes that a brush head be produced by injection molding; the hair density should be between 200 and 400 hairs per square centimeter.

Reference is also made to the fact that gynecological sample removal instruments are also known from DE 83 20 917 U1, U.S. Pat. App. Pub. No. 2009/0062691 A1, U.S. Pat. App. Pub. No. 2014/0083213 A1, WO 2016/104 779 A1, and KR 101 504 264 B1. In particular, KR 101 504 264 B1 discloses a detachable medical brush that allows the user, for the diagnosis of precursors of cervical cancer, to insert the brush in order to collect mucous material, where the brush can be separated from a handle and can be placed in a container. At the caudal end, the brush is formed with a cup-shaped region of fibers, the ends of which point in the cranial direction and slightly outward. This cup-shaped region is adjoined in the cranial direction by a region with bristles which extend radially outward, have a shorter length than those of the cup-shaped region and narrow generally conically toward the cranial end. The brush is formed by a pair of twisted wires between which the bristles are held. With their relatively long length, the bristles of the proximal region give the impression of a continuous lateral surface, which is effected by the bristles spreading away from the brush axis formed by the twisted wires.

### SUMMARY

A comparable brush was also used over a long period of time by the inventor of the present invention. It was found however that the aforementioned problems are not yet solved in a way that is entirely satisfactory in all circumstances, that is to say with respect to the problems concerning simple intervention, safe collection of sample material in a wide variety of anatomical conditions, and usability with a large number of different laboratory methods without the need to use different sample collection assemblies for different methods.

It is therefore desirable to make available an improved sample removal assembly for gynecological examinations of the vagina or cervix, in particular for gynecological preventive examinations, which assembly at least partially solves at least some of the aforementioned problems and yet permits cost-effective production.

An object of the present invention is to provide new material for industrial application.

The invention thus proposes among other things that, in a gynecological sample removal instrument for vaginal or cervical examinations, which at the cranial end has a multiplicity of bristles that are fixed to a wire assembly that extends in an axial direction, provision is made that the bristles are formed of at least 600 plastic filaments with a diameter of between 350 μm and 900 μm and with an axial line density of at least 600 filaments over a maximum axial extent of 3 cm.

A first concept of the present invention is therefore the discovery that a very large number of sample removal filaments of sufficient density afford advantages both in terms of the use for smear tests and also in terms of the transfer of collected material into a liquid. This was not wholly expected. Although the comparatively thin bristles each takes up only a small amount of material and although, irrespective of the large number of plastic bristles, only relatively little material is collected overall, a highly suitable sample removal instrument is nonetheless obtained, since the materials collected during the sample removal can be recovered from the sample removal instrument very efficiently, substantially better on the whole than in the prior art.

It can be advantageous if the bristle density is not excessively high, i.e., the bristles are not too thin and are not too close together. If the filaments are too thin, then too little material is collected from the corresponding free-standing bristles. By contrast, if very thin bristles lie close together, capillary effects in the spaces between bristles are too strong. This can lead to different material being collected than would be the case without capillary effects, since, on the one hand, the bristles adhering to each other by capillary action become somewhat stiffer and, on the other hand, especially fine material tends to remain adhering to the bristles in the examining laboratory, i.e., a different fraction of the collected cells is examined. It is therefore preferable if the bristle density is limited. If the axial extent of the bristle-carrying region is 3 cm, for example, it is then preferable if the total number of filaments remains below 1800, preferably below 1500 and particularly preferably below 1200. Total filament counts can clearly be derived from these statements for other axial extents. It will be clear from the above explanation that slightly thicker filaments counteract the capillary effect and that, accordingly, the maximum expedient bristle density can be dependent on thickness. It will also be clear that the expedient total filament count will depend on the axial length along which bristles are provided. From the above explanations, it will be gathered that the density should not be too high.

When preparing specimens for microscopy, which requires thin application of the material (monolayer) to a slide, it is advantageous if only a little material is present on each of the thin bristles, since this makes it easier to produce thin smears for the microscopy. When transferring the material into a liquid, account must be taken of the fact that, in the gynecological examination, it is not just the desired cells to be examined that are generally removed; instead these cells will be embedded in a matrix of other material such as mucus or the like. For laboratory tests, it is then necessary to remove the cells from this material. If a subsequent examination process requires the use of solvents in which the matrix material removed with the cells forms lumps, for example by coagulation or the like, the recovery of the cells is extremely difficult.

That the invention is advantageous is attributed, at least on the basis of our current understanding, to the fact that it is not only a few large, very compact lumps that are formed, but instead at any rate a very large number of small lumps in which cells etc. are either not so strongly fixed, as a result of which the cells present at the surface can be easily separated, or else, on account of the small lump size, a smaller fraction of cells is completely enclosed in their interior. At the same time, the design of the brush permits the recovery from the preferred sites.

The overall recovery of examination material with the gynecological sample removal instrument is thus considerably improved. Moreover, by using suitably thin filaments in contrast to hard spatulas, instruments with coarse bristles or scrapers, there is much less tendency for the removal of sample material to cause pain or irritation. In contrast to wooden and/or plastic spatulas, foam instruments or polyethylene instruments with a small number of very broad “small teeth,” the instruments according to the invention are also suitable, by virtue of their structure, to be inserted into the histologically formed deep cervical crypts and to collect the cylindrical epithelial cell material lying deep therein. Moreover, on account of their small diameter, the bristles are still so movable that bleeding at the sample removal site, or on the way to the latter when inserting the sample removal instrument, can be largely avoided, in most cases completely avoided. The use of the sample removal instrument is thus much more comfortable for a patient.

For the physician who has to insert the gynecological sample removal instrument, the use of the instrument is also made considerably easier because there is less negative feedback from the patients. This also makes it easier to become familiar with the handling of the gynecological sample removal instrument according to the invention, especially since it is suitable for removing samples for a large number of different examinations and, therefore, initially inexperienced physicians also gain experience more quickly.

This even applies where different brush shapes are intended to be used for vaginal and cervical brushes but where both, i.e., vaginal brush and also cervical brush, are intended to be used for collecting material for different subsequent examination methods.

It is possible and advantageous if, in a gynecological sample removal instrument, provision is further and/or additionally made that the plastic filaments are produced from polyamide. Polyamide filaments have good physiological tolerability and are inexpensive. They are available in a large number of different diameters, are inexpensive and, in the strengths indicated according to the invention, offer a flexibility that is ideal for collecting cells at the required sites.

It is possible for the plastic filaments to be separated mechanically. It is possible and advantageous if, in a gynecological sample removal instrument according to the invention, provision is made in particular that the plastic filaments are cut off or pinched off or sheared off.

It is possible in particular that they are already cut to the length required for the particular position within the overall brush before they are fixed to the bristle body, which reduces waste. Alternatively, trimming can be carried out by first of all producing a bristle body with bristles of approximately equal length and then cutting the bristles to the required length. This is at present preferred. In each case, the cut edges afford advantages in the uptake of cell material since, in contrast for example to cut edges obtained by separation by laser cutting and the like, they are able to engage particularly well with the sample removal site in order there to take up the cells that are to be collected. All the same, it would be possible to use laser cutting or the like.

It is possible and advantageous if, in a gynecological sample removal instrument, provision is further and/or additionally made that the plastic filaments have a mean diameter of between at least 500 μm, preferably at least 600 μm, and at most 850 μm, preferably at most 800 μm.

These diameters are preferred specifically in the case of polyamide or aramid fibers, because they offer sufficient stiffness for entraining cell material when the bristles are moved along the cervical or vaginal wall, but they are not so inflexible as to cause injuries. It will be noted that the bristles will typically protrude perpendicularly with respect to the axis directly after production and will have to be removed from this radial orientation in order to provide the preferred cup shape of the outer bristles, which in particular permits better sample removal and better visual monitoring. This can be done by heat treatment for example, although it typically suffices if the brushes are introduced at the production site into a narrow tube. The typical times leading up to use are then sufficient to ensure the cup shape.

It is possible and advantageous if, in a gynecological sample removal instrument, provision is further and/or additionally made that at least 750 bristle ends are present over a maximum axial extent of 3 cm. This line density does not cause mutual obstruction of the points during the sample removal and also makes available a sufficiently high number of tapping points. At the same time, it is preferable if the number of tapping points used is not too high, so as to avoid capillary effects and the like. In example preferable embodiments, no more than 1800 tapping points are used over an axial extent of 3 cm, particularly preferably fewer than 1500 tapping points, more preferably fewer than 1200 tapping points, and particularly more preferably fewer than 1100 tapping points. It is mentioned for the sake of completeness that, with a shorter axial length, the number of tapping points will accordingly be reduced.

It is possible and advantageous if, in a gynecological sample removal instrument, provision is further and/or additionally made that the bristles, with the wire assembly fixing them, are arranged as an end region, that can be broken off after the examination, on a flexible plastic rod. In a preferred variant, the wire assembly, which typically fixes the bristles by the twisting together of two wire elements, is arranged together with the bristles on a plastic rod. It is particularly preferable if the end region in which the wire assembly fixing the bristles is arranged is to be broken off from a flexible plastic rod of sufficient flexibility intended for advancing as far as the removal site. Breaking it off immediately ensures that the sample removal instruments can be used only in disposable-type methods since, after a given examination and after the tip has then been broken off by manual actuation or the like, they could not then be inserted again into the vagina. The predetermined breaking point can be worked into the plastic rod, specifically in such a way that the breaking off can be effected by hand, without using a tool, or the breaking off can be effected on an edge, in particular on the edge of a container provided for transport to the laboratory. The predetermined breaking point can be easily dimensioned such that breaking off inside the body need not be feared, but such that manual breaking off is still easily permitted. In this context, it will be noted that no objects typically lie on the way to the sampling site in gynecological examinations, and therefore no great mechanical stresses are expected to be placed on the stem or shaft.

It is possible and advantageous if, in a gynecological sample removal instrument, provision is further and/or additionally made that all of the bristles have the same nominal diameter. This is particularly advantageous especially from the point of view of manufacturing technology. Alternatively, however, it would also be possible to fix tufts of bristles of varying diameter by means of the wire or to use different thicknesses along the twisting thereof.

In one variant, the gynecological sample removal instrument is a vaginal brush. Vaginal smears are performed especially in postoperative tumor screening, but also preventively. The vaginal brush of the present invention is particularly suitable for the latter. It is suitable in particular for cytological examinations, HPV tests and molecular biological tests. For these purposes, use has hitherto been made of wooden spatulas or rounded plastic spatulas that patients find rather uncomfortable during use. With the vaginal brush proposed here, which has a multiplicity of several 100 tapping points, there is by comparison a considerable improvement in the pre-analytical sensitivity, and it at the same time allows smears to be taken from sites that are difficult to access. Especially in situations of tumor aftercare after radiotherapy and very much so after so-called side-to-side colpotomy closure, it is often the case that the vaginal cul-de-sac (following hysterectomy) is not spherical at all but often drawn out unevenly and asymmetrically, such that “retention pockets” arise. However, these too can be easily reached by the bristles, which is significant since it is here that a so-called local recurrence often has its origin. The cell removal can also be done gently, which is particularly advantageous especially in the atrophic conditions that are typically present in this population.

This vaginal brush is then preferably dimensioned and arranged for the insertion into the vagina and for the removal of samples from the vagina. For this purpose, in an example embodiment, the vaginal brush is designed such that the bristles provide a cranially rounded brush contour, preferably with a caudal diameter away from the center of between 20 mm and 29 mm, particularly preferably of between 24 mm and 28 mm.

It is alternatively possible that the gynecological sample removal instrument is a cervical brush in which a cup-shaped bristle region is present at the caudal end, and the bristles of which protrude obliquely with respect to the central axis and extend up to a diameter of at most 28 mm, preferably 26 mm, transversely with respect to the axis, where the cup-shaped bristle region has an outer ring of long bristles and, enclosed by the latter, at least one inner ring of bristles, where preferably the bristles of the outer bristle ring are longer than the bristles of the inner bristle ring at least by such an extent that at least a substantial part of the outer bristles extends axially farther forward than the bristles of the inner bristle ring during the phase of insertion into the patient. With respect to the question of from when the proportion of the outer bristles that end nearer the insertion end of the sample removal instrument than the bristles of the inner bristle ring can be regarded as substantial, it will be noted that the cervical brush is typically inserted with monitoring through a speculum, and the bristle central region surrounded by the bristle cup is intended to be pushed into the cervix for collection of cells. It is then particularly clearly visible that the cup-forming bristles are spread further open and come to bear on the outer regions of the cervix. Against this background, the proportion of bristles extending far forward is at any rate substantial if it is clearly discernible when this is the case. The preferred length of the outer bristles then ensures that sample material is obtained from locations lying sufficiently far from the cervical mouth, independently of the anatomy as it were, while at the same time excellent visual monitoring of the depth of insertion is ensured by the bristle deformation. No tactile detection of the bristle deformation is therefore needed, and instead a deformation can also be observed visually. When the brush is moved out after the sample has been obtained by rotation, the cup shape will then reform.

In the case of a cervical brush, the preferred cup shape ensures an optimal uptake of cells, where the collection points can respond at different times. Thus, contact can first be made in the region of the outer bristles and, when the outer cup bristles have come into contact, the inner bristles of the cup-shaped region are also brought into contact, typically nearer the cervical mouth. The brush shape here not only provides a nominally high number of contacts, it also ensures that a reasonable amount of cell material can be taken up on each of the bristles and that the filaments do not appreciably obstruct each other in the uptake.

The cervical brush is preferably continued by a short-bristle region in the direction cranially from the cup-shaped bristle region. In the case of the cervical brush, it is advantageous if a short-bristle region insertable as far as the region of the ectocervix is arranged cranially with respect to the cup-shaped bristle region, having a diameter in such a way that the diameter of the outer bristle ring in the cup region is at least 4 times, preferably at least 5 times, particularly between 5.5 and 7 times greater than the diameter of the short-bristle region, where the short bristles protrude generally radially from the axis, and the short bristles form a helix structure with windings spaced apart from one another. The term “generally radially” is used here because, for production-related reasons, it cannot be guaranteed that the short bristles protrude exactly perpendicularly from the central axis. However, it will be evident to a person skilled in the art that slight deviations will occur for example when “twisting” the short bristles in between two wire segments during the production of the brush.

In a particularly preferred variant, over 200 filament tapping points can be formed by the bristles in the region of the cup-forming bristles, for example by over 240 bristles. These represent cervix tapping points. The stated diameters are also greater than the maximum ectopy diameter of 22 mm determined in morphometric studies, cf. the studies by Przybora L A, Plutowa A. “Histological topography of carcinoma in situ of the cervix uteri,” Cancer. 1959 March-April; 12(2):263-77 or Jacobson D L, Peralta E, Farmer M, Graham N M, Wright T C, Zenilman J., “Cervical ectopy and the transformation zone measured by computerized planimetry in adolescents,” Int. J. Gynaecol Obstet. 1999 July; 66(1):7-17.

Preferably over 500 filament tapping points, preferably between 500 and 700, particularly preferably more than 550 and/or fewer than 600, can be present in the cup region.

With the cervical brush, in particular in the preferred embodiments, a defined depth of insertion sufficient for the vast majority of all patients is not only attainable but also easily reproducible, which permits particularly simple and standardized removal of samples. In this way, irrespective of the short bristles, an unpleasant sensation during the insertion of the bristles is additionally avoided or at least reduced. Since the cervical brush has to be inserted until contact with the ectocervix, excessively high endocervical sampling is at the same time avoided, where the thin brush hairs lead to cell recovery that is also gentle on the patient and that also entails little bleeding, which is clearly advantageous both for the patient and for the subsequent laboratory examinations.

In the gynecological sample removal instrument, it is moreover and/or alternatively possible that a small ball of plastic is present at the cranial end of the wire, in particular made of a plastic material applied in the form of a liquid drop to the wire end and cured on the wire end. This offers protection and is also helpful during the insertion, particularly of the cervical brush, into the rather narrow juvenile cervical channels or into ones showing age-related atrophy. In this way, irrespective of the short bristles, an unpleasant sensation during the insertion of the bristles is avoided or at least reduced. It will be noted that such a drop of plastic material is advantageous in particular for the cervical brush.

The invention is described below only by way of example, and with reference to the drawings.

### BRIEF DESCRIPTION OF THE DRAWINGS

FIG. 1 shows a perspective view of a cervical brush according to an example embodiment of the present invention.

FIG. 2 shows the side view of the cervical brush from FIG. 1, according to an example embodiment of the present invention.

FIG. 3 shows a perspective view of a vaginal brush according to an example embodiment of the present invention.

### DETAILED DESCRIPTION

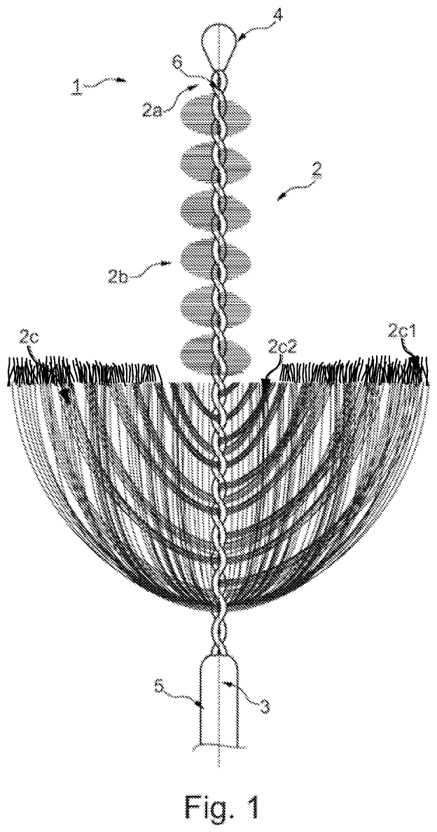

According to FIG. 1, a gynecological sample removal assembly 1, designated overall by 1, comprises at the cranial end a multiplicity of bristles which are fixed to a wire assembly that extends in an axial direction, where the bristles are formed of at least 600 plastic filaments with a diameter of between 350 μm and 900 μm and with an axial line density of at least 600 filaments over a maximum axial extent of 3 cm.

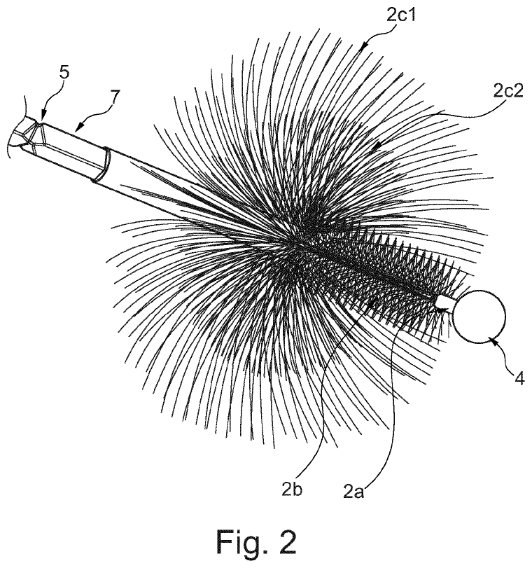

In the illustrative embodiment shown in FIGS. 1 and 2, the gynecological sample removal instrument 1 is a cervical brush for removal of material from the region of the mouth of the cervix, in particular also from the outer region directly around the mouth of the cervix. The cervical brush shown can be received in a tube which is dimensioned such that the longer bristles, initially still protruding radially after production, adopt the desired cup shape during transport to the physician or during storage, for which purpose the initially still radially protruding longer bristles are bent within the tube.

In the embodiments shown, the plastic filaments are fixed between two wire segments that are twisted together along the brush axis, wherein bristle portions are brought between the wire segments and the wires are then continuously turned about each other. By virtue of this fixing, it is easily possible to obtain the desired high line densities by adapting the twisting strength, the gradient along the axis, the density of the bristles between the wires, etc., and also to achieve the desired total numbers of filaments or bristles of a brush according to the invention.

In the present case, the plastic filaments are produced from polyamide material (known by the trade name nylon).

The bristles, in the present case after they have been fixed in the twisted wire segments, are cut to length in such a way that, for the illustrated gynecological sample removal instrument of the present invention as a cervical brush, a brush structure is obtained that can be seen to have three different regions, namely, from cranial to caudal, a bristle-free proximal end, i.e., an end facing the patient interior during use, a short-bristle region for insertion into the endocervix, and a long bristle region for removing sample material from the region of the ectocervix. This can be seen particularly clearly in FIG. 2. At the upper bristle-free end, a drop of plastic material is dropped over the twisted-together wire segments, among other reasons in order to protect the patient from injuries caused by the otherwise pointed end. It will be noted in this connection that the two wire segments can be formed along one and the same continuous integral wire, which is bent back on itself at the tip.

In the caudal direction from the tip, a portion having in this case about 240 short bristles and a clear helix structure is first of all provided. The bristles have a diameter of between 600 μm and 800 μm and a length away from the axial center of about 2 mm, i.e., they form a helix with a diameter of ca. 4 mm. The clearly discernible helix structure of the bristles, which leads to axial interspaces between the bristle ends, has the effect that the brush can be easily inserted with rotation into the cervix, which permits more sensitive treatment, and moreover at the same time ensures that cell material present on the bristles remains separate, at least where there is only slight entrainment of mucus. Removal of sample material from the region of entry into the cervix is also permitted.

A long-bristle region is provided nearer the grip part of the shaft, i.e., in the caudal direction from the short-bristle region. The long bristles of the long-bristle region extend forward in a cup shape away from the shaft, like the head of a tulip, which opens out in a cup shape away from its stalk, where the outer bristles extend farther forward in the cranial direction than the inner bristles. The cup formation can be achieved when, after the nylon filaments have been twisted in between the wire segments that are to be twisted together, the bristles that are cut to the appropriate length are cast, with the caudal end of the wire segments (the end facing the physician), into a plastic rod by which the instrument can be gripped and pushed to the site, and the rod is then received with the bristle portion in a transport tube that has a diameter smaller than the diameter of the inner bristles, specifically by first pushing the caudal end into the transport tube, the latter having a diameter much smaller than the length of the caudal bristles. When the assembly is pushed, with the shaft at the front, into the tube, the longer nylon filaments are bent cranially, which results in the desired cup shape being obtained solely as a result of the time that is needed between production and transport to the physician who uses the brush.

As will be seen from FIG. 1, the outer cup filaments are longer than the inner ones. In the preferred variant, about 560 filament points are provided in the cup region. In the illustrative embodiment, the filaments provide a diameter of 24 mm in the inner cup region and of about 28 mm in the outer region. These diameters can of course be easily varied, e.g., by ±10 to 15%, although the diameters indicated have proven expedient in practice.

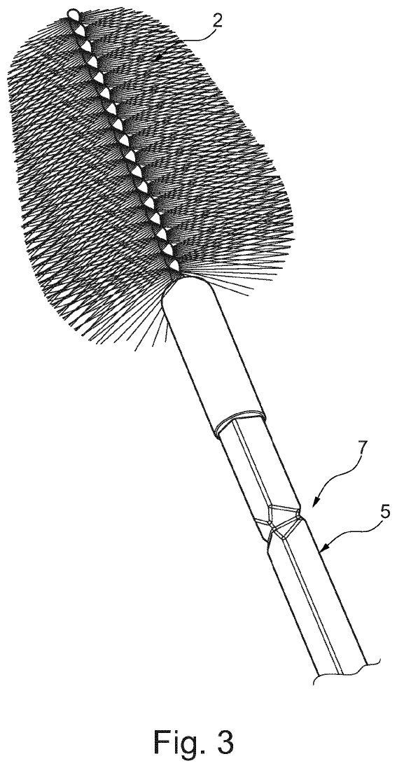

As can be seen from the thus far identical second illustrative embodiment in FIG. 3, the shaft assembly is formed with a predetermined breaking point close to the bristle region, although the predetermined breaking point is so far from the bristle region that, in order to break off the bristle end from the grip part of the shaft, a sufficient region still remains at which the bristle end can be grasped. This subregion remaining at the bristle end after separation is also sufficient for the manipulation that is needed in the laboratory for removing the collected sample material from the brush. The shaft is also dimensioned such that its caudal end still remains outside the vagina, even when used in patients with a long vagina. Near the caudal end, the shaft can in particular be completely round, in order to make it easier to rotate one way or back and forth between thumb and index finger.

During use, the gynecological sample removal instrument is guided to the site while viewing through a speculum. The arrival at the site, i.e., at the correct depth of insertion, can be easily detected visually by observation of the deformation of the cup-forming bristles. This is possible with the naked eye alone, although it can also be done by coloscopy. The gynecological sample removal instrument is then moved in rotation at the site, before being pulled back out again. By doing this, sample material that is to be removed gathers on the bristles. The flexibility of the bristles at the same time ensures that material is gathered from those layers that are particularly relevant for typical examinations. These are very thin tissue layers. In addition to the prevention of cervical cancer in which the HP virus has a key causal role, the smear collection instrument can also be used to collect material for other microbiological tests, for example for chlamydia, trichomoniasis, gonococci, herpes simplex, A-streptococcus, Treponema pallidum, etc. Additionally, single instrument can be used on its own to collect sample material simultaneously from the regions that are particularly relevant to the laboratory examinations, i.e., from the endocervix and ectocervix, and from the particularly significant layers there, and, what is more, untrained personnel are also able to ascertain the correct depth of insertion by simple observation of a readily visible deformation.

The brush can then be removed from the patient's body and the cells can be smeared on a glass slide. If so desired for the subsequent tests, the front end near the brush can be broken off, specifically by being introduced into a transport container and being left in the container by being buckled. After suitable labeling, etc., it can be transported to a laboratory where the collected samples are examined. If this is done by smearing the sample onto a microscope slide, the fine distribution of the collected sample on the many filaments results in a thin layer that is easy to view under a microscope. By contrast, if the sample material has to be transferred into a solution for further (cytological or other) examinations, this is likewise possible without any problems.

A further illustrative embodiment is shown in FIG. 3. The gynecological sample removal instrument shown there is a vaginal brush, which has a brush shape that is optimized for removal of vaginal sample material. For this purpose, the vaginal brush of FIG. 3 has a generally cone-shaped bristle region, which tapers to a comparatively blunt shape at the cranial end, i.e., the proximal bristles still form a diameter of over 5 mm or, in the illustrative embodiment shown, of even 10 mm.

This also dispenses with the need to apply a drop of plastic to the caudal end. Particularly if the wire elements clamping and fixing the bristles are formed in one piece from a continuous segment of wire, which is bent back on itself at the proximal end with a sufficiently large radius of curvature, specifically in combination with the still sufficiently long proximal bristles, damage to the vaginal wall is avoided. However, the presence of a drop of plastic is typically preferred also in the vaginal brush, since the risk of injury is reduced even in the case of production errors. In the case of the cervical brush too, it is possible and preferable that the wire elements clamping and fixing the bristles are formed in one piece from a continuous segment of wire, which is bent back on itself at the proximal end with a sufficiently large radius of curvature. There, however, on account of the small bristle diameters of the proximal helix region, the presence of the additional drop of plastic is more clearly preferable than in the case of the vaginal brush, which at least in the illustrative embodiment is shown without a drop of plastic.

The long bristles provided at the cranial region of the vaginal brush prevent injury to the vaginal wall during insertion, even in the hands of a clumsy physician.

Over the total range of the bristle region, which extends axially by about 20 mm, there are 800 filament attachment points in the illustrative embodiment shown, these being distributed more or less uniformly about the circumference. Efficient removal of sample material is thus achieved, similarly to the case of the cervical brush, and also efficient deposition of the removed sample material into solution or onto a microscope slide for examination in a laboratory is achieved.

It will be noted that the instrument can readily be produced in a cost-effective manner and also that bristles made of a material other than nylon can be used.

### LIST OF REFERENCE SIGNS

1 gynecological sample removal instrument
2 bristles
2a bristle-free proximal end, i.e., the end facing the patient interior during use
2b short-bristle region for insertion into the endocervix
2c cup-shaped bristle region for sample removal from the region of the ectocervix
2c1 outer cup filaments (FIG. 2)
2c2 inner cup filaments (FIG. 2)
3 brush axis
4 drop
5 stem
6 wire segments
7 predetermined breaking point (FIG. 3)

1-10. (canceled)

11. A gynecological cervical or vaginal sample removal instrument comprising:
a wire assembly that extends in an axial direction; and
at a cranial end of the sample removal instrument, a multiplicity of bristles that are fixed to the wire assembly, wherein the bristles are formed of at least 600 plastic filaments with a diameter of between 350 μm and 900 μm and with an axial line density of at least 600 filaments over a maximum axial extent of 3 cm.

12. The sample removal instrument of claim 11, wherein the plastic filaments are produced from polyamide.

13. The sample removal instrument of claim 12, wherein the plastic filaments are cut off or sheared off mechanically.

14. The sample removal instrument of claim 13, wherein the plastic filaments have a mean diameter of between at least 600 μm and at most 800 μm.

15. The sample removal instrument of claim 13, wherein the plastic filaments have a mean diameter of between at least 500 μm and at most 850 μm.

16. The sample removal instrument of claim 15, wherein at least 750 bristle ends are present over a maximum axial extent of 3 cm.

17. The sample removal instrument of claim 16, wherein all of the bristles have a same nominal diameter.

18. The sample removal instrument of claim 11, wherein:
the bristles, with the wire assembly to which they are fixed, are arranged as an end region of a flexible rod; and
the end region can be broken off from the flexible rod after an examination.

19. A vaginal brush comprising:
a wire assembly that extends in an axial direction; and
at a cranial end of the vaginal brush, a multiplicity of bristles that are fixed to the wire assembly;
wherein:
the bristles (a) are formed of at least 600 plastic filaments with a diameter of between 350 μm and 900 μm and with an axial line density of at least 600 filaments over a maximum axial extent of 3 cm and (b) are arranged and structured so that they provide the vaginal brush with a cranially rounded brush contour; and
the plastic filaments are produced from polyamide, are cut off or sheared off mechanically, and have a mean diameter of between at least 500 μm and at most 850 μm.

20. The vaginal brush of claim 19, wherein the brush contour has a caudal diameter away from the center of between 20 and 29 mm.

21. The vaginal brush of claim 19, wherein the brush contour has a caudal diameter away from the center of between 24 and 28 mm.

22. A cervical brush comprising:
a wire assembly that extends in an axial direction; and
at a cranial end of the vaginal brush, a multiplicity of bristles that are fixed to the wire assembly;
wherein:
the bristles (a) are formed of at least 600 plastic filaments with a diameter of between 350 μm and 900 μm and with an axial line density of at least 600 filaments over a maximum axial extent of 3 cm, and (b) form a cup-shaped bristle region at a caudal end of the cervical brush;
the plastic filaments are produced from polyamide, are cut off or sheared off mechanically, and have a mean diameter of between at least 500 μm and at most 850 μm;
in the cup-shaped bristle region, the bristles:
protrude obliquely with respect to a central axis of the cervical brush and extend up to a diameter of 28 mm transversely relative to the axis; and
form at least one inner ring of bristles and an outer ring of long bristles that enclose the at least one inner ring of bristles.

23. The cervical brush of claim 22, wherein, in the cup-shaped bristle region, the bristles extend no more than up to a diameter of 26 mm transversely relative to the axis.

24. The cervical brush of claim 22, wherein the bristles of the outer ring are longer than the bristles of the inner ring by at least such an extent that at least a substantial part of the bristles of the outer ring end caudally with respect to the bristles of the inner ring.

25. The cervical brush of claim 22, wherein:
a short-bristle region of short ones of the bristles that is insertable as far as a region of an ectocervix is arranged cranially with respect to the cup-shaped bristle region;
the diameter of the outer ring in the cup region is at least 4 times greater than the diameter of the short-bristle region, the short ones of the bristles protrude generally radially from the axis, and the short bristles form a helix structure with windings spaced apart from one another; and/or
at a proximal end of the wire assembly, a wire end is provided with a plastic protector.

26. The cervical brush of claim 25, wherein the diameter of the outer ring in the cup region is at least 5 times greater than the diameter of the short-bristle region.

27. The cervical brush of claim 25, wherein the diameter of the outer ring in the cup region is between 5.5 and 7 times greater than the diameter of the short-bristle region.

28. The cervical brush of claim 25, wherein, at the proximal end of the wire assemble, the wire end is provided with the plastic protector, and the plastic protector is made of a plastic material applied in the form of a liquid drop to the wire end and cured on the wire end.

29. A gynecological cervical sample removal brush comprising:
a wire assembly that extends in an axial direction; and
at a cranial end of the brush, a plurality of bristles that are fixed to the wire assembly;
wherein:
the bristles are formed of at least 600 plastic filaments with a diameter of between 350 μm and 900 μm and with an axial line density of at least 600 filaments over a maximum axial extent of 3 cm;
the bristles include short bristles forming a short-bristle region;
the bristles include a cup region including an outer ring of the bristles; and
one or both of the following features (a) and (b):
a diameter of the outer ring is at least 4 times greater than a diameter of the short-bristle region, the short bristles protrude generally radially from a central axis of the brush, and the short bristles form a helix structure with windings spaced apart from one another; and
at a proximal end of the wire assembly, a plastic protector is provided on a wire end of the wire assembly.

30. The brush of claim 29, wherein the diameter of the outer ring is at least 5 times greater than the diameter of the short-bristle region.

31. The brush of claim 29, wherein the diameter of the outer ring is between 5.5 and 7 times greater than the diameter of the short-bristle region.

32. The brush of claim 29, wherein the plastic filaments are produced from polyamide.

33. The brush of claim 32, wherein the plastic filaments are cut off or sheared off mechanically.

34. The brush of claim 33, wherein the plastic filaments have a mean diameter of between at least 600 μm and at most 800 μm.

35. The brush of claim 33, wherein the plastic filaments have a mean diameter of between at least 500 μm and at most 850 μm.

36. The brush of claim 35, wherein at least 750 bristle ends are present over a maximum axial extent of 3 cm.

37. The brush of claim 36, wherein all of the bristles have a same nominal diameter.

38. The brush of claim 37, wherein:
the bristles, with the wire assembly to which they are fixed, are arranged as an end region of a flexible rod; and
the end region can be broken off from the flexible rod after an examination.

39. The brush of claim 38, wherein:
the cup region further includes an inner ring of the bristles;
the bristles of the outer ring are longer than the bristles of the inner ring by at least such an extent that at least a substantial part of the bristles of the outer ring ends caudally relative to the bristles of the inner ring.
